(19) United States
(12) Patent Application Publication
(10) Pub. No.: US 2014/0134321 A1
(43) Pub. Date: May 15, 2014

(54) NITRIC OXIDE-RELEASING COATINGS

(71) Applicants: The University of North Carolina at Chapel Hill, Chapel Hill, NC (US); Novan, Inc., Durham, NC (US)

(72) Inventors: Nathan Stasko, Durham, NC (US); Mark Schoenfisch, Chapel Hill, NC (US); Benjamin Privett, Saxapahaw, NC (US); Jae Ho Shin, Seoul (KR); Wesley Storm, Carrboro, NC (US)

(73) Assignees: The University of North Carolina at Chapel Hill, Chapel Hill, NC (US); Novan, Inc., Durham, NC (US)

(21) Appl. No.: 14/022,717

(22) Filed: Sep. 10, 2013

Related U.S. Application Data
(62) Division of application No. 12/903,614, filed on Oct. 13, 2010, now abandoned.
(60) Provisional application No. 61/251,133, filed on Oct. 13, 2009.

(51) Int. Cl. A61K 33/00

(52) U.S. Cl. CPC A61K 33/00
USPC 427/2.24

(57) ABSTRACT

Provided according to embodiments of the invention are NO-releasing sol-gel coating formed from a sol precursor solution comprising a backbone alkoxysilane and a diazeniumdiolate-modified alkoxysilane. Further provided are methods of producing NO-releasing sol-gel coatings. Such methods may include (a) co-condensing a sol precursor solution comprising a backbone alkoxysilane and a diazeniumdiolate-modified alkoxysilane in a solvent to form a sol; (b) coating a substrate with the sol; and (c) drying the sol to form the NO-releasing sol-gel coating.

### CROSS-REFERENCE TO RELATED APPLICATIONS

This application claims priority from U.S. Provisional Application Ser. No. 61/251,133, filed Oct. 13, 2009, the disclosure of which is hereby incorporated by reference herein in its entirety

### STATEMENT REGARDING FEDERALLY SPONSORED RESEARCH OR DEVELOPMENT

This invention was made with United States Government support under Grant No. 07-4569, awarded by the National Institute of Health. The United States Government may have certain rights in the invention.

### FIELD OF THE INVENTION

The present invention is related to coating materials. More specifically, the present invention is related to nitric oxide-releasing coating materials which may be applied to various substrates. The present invention also relates to methods of coating materials,

### BACKGROUND OF THE INVENTION

Currently, over thirty-five million Americans undergo surgical procedures involving artificial implants each year. Medical implants and devices are used in every organ of the body and constitute a $27.9 billion industry. This figure is expected to climb even higher in the future as the elderly population continues to grow in number. Although medical implants and devices are widely used, there are a number of associated risks stemming from the body's response to foreign materials.

This response is a complex physiological cascade resulting from the body's attempt to defend against invasion by the foreign material. Upon implantation of a medical device, proteins may be adsorbed on the device surface and inflammatory cells may be recruited, with neutrophils and macrophages modulating the host response. Subsequently, foreign body giant cells may be formed from the macrophages and may remain at the surface of the device indefinitely, secreting degradative agents and causing localized damage and often chronic inflammation. The foreign body giant cells may secrete cytokines that trigger fibroblasts to deposit a fibrous capsule around the device, consisting of an avascular layer of collagen, which may effectively isolate the device from host tissue. As a result, tissue integration may be ineffective because the device may be unable to actively interact with surrounding tissue. Such responses can lead to chronic pain and, ultimately, rejection of the device.

Bacterial infection is also of concern, as bacteria may be found in nearly 90% of all implantation sites immediately following surgery. The bacteria may lead to the formation of biofilms, which can cause chronic illness with generalized symptoms, such as headache, nausea, vomiting, abdominal cramps, sore throat, sore eyes, and fever, that may make an accurate diagnosis difficult. Although the incidence of infection associated with medical devices is relatively low, the associated morbidity and mortality rates are high. Further, the costs of addressing device infections can be five to seven times the initial cost of the implantation. See Higashi & Marchant, “Implant Infections,” in Von Recum & Jacobi, Biomaterials Evaluation 493 (1999). Localized methods, including antibiotic-doped bone cements and wound irrigation with antibiotic solutions, have emerged to address the prevention and treatment of infected implant sites. However, infected implant sites still exist.

Previous research has identified nitric oxide (NO) as a promising candidate for addressing concerns of ineffective tissue integration, fibrous encapsulation and bacterial infection. NO is a highly reactive gas with many biological functions. See, for example, Fang, Nitric Oxide and Infection (1999); Loscalzo et al., Nitric Oxide and the Cardiovascular System (2000); Wang et al, (ed.), Nitric Oxide Donors: for Pharmaceutical and Biological Applications (2006); and Packer et al., Nitric Oxide: Part C, Biological and Antioxidant Activities (1999). Due to its ability to inhibit the aggregation of platelets and adhesion of leukocytes, reduce smooth muscle proliferation, and decrease inflammation, NO may be able to prevent or treat complications such as restenosis and thrombus formation, which can result from contact with synthetic medical devices. Further, NO possesses mechanistically complex antimicrobial activity against a broad range of parasitic, fungal, bacterial, and viral pathogens. These attributes suggest that it would be desirable to provide a localized, persistent concentration of NO in the vicinity of an invasive medical device upon implantation.

Sol-gel coatings capable of NO release have also been prepared previously. However, these previous strategies for the preparation of NO-releasing sol gel-based materials typically involve coating the surface of a substrate with a non-NO modified siloxane monolayer or xerogel network, followed by NO modification of the entire substrate using high pressures of NO. This method limits the size and shape of substrate to the dimensions of the high pressure chamber used to introduce the NO and also may reduce the number of sites that can be modified with NO, as many of the silane precursor structures may be buried within the coating and inaccessible to reaction with the NO. Additionally, not all devices, especially those with electronic components or sensitive sensor membranes can withstand the harsh exposure conditions required to load NO following surface modification. The extreme pressures or basic conditions required may lead to degradation or device failure.

### SUMMARY OF THE INVENTION

Provided according to some embodiments of the invention are NO-releasing sol-gel coatings formed from a sol precursor solution that includes a backbone alkoxysilane and a diazeniumdiolate-modified alkoxysilane. Additionally, in some embodiments, the sol precursor solution includes a multifunctional alkoxysilane that includes at least one functionality that provides to the sol-gel coating at least one additional property such as anti-corrosive activity, anti-inflammatory activity, anti-microbial activity, anti-oxidative activity, additional cross-linking functionality, surface charge, hydrophilicity and/or hydrophobicity. In some embodiments, the multifunctional alkoxysilane has the formula R′R″R′″SiOR, wherein R is H, alkyl or substituted alkyl, R′, R″ and R′″ are each independently a substituted or unsubstituted alkyl, a substituted or unsubstituted aryl, a substituted or unsubstituted alkylaryl, a substituted or unsubstituted arylalkyl or an organic moiety that provides at least one additional property to the sol-gel coating, wherein at least one or R′, R″ and R′″ is an organic moiety that provides at least one additional property to the sol-gel coating.

According to some embodiments, the NO-releasing sol-gel coatings may have excellent NO storage capability. For example, in some embodiments, the coating has an NO storage greater than 0.01 μmol NO·cm⁻².

Also provided according to some embodiments are substrates coated with at least one layer of a sol-gel coating according to an embodiment of the invention. In some embodiments, the substrate is a medical device and, in some embodiments, the medical device includes a metallic surface.

Furthermore, in some embodiments of the invention, provided are methods of forming NO-releasing sol-gel coatings that include (a) co-condensing a sol precursor solution including a backbone alkoxysilane and a diazeniumdiolate-modified alkoxysilane, and optionally a multifunctional alkoxysilane, in a solvent to form a sol; (b) coating a substrate with the sol; and (c) drying the sol to form the NO-releasing sol-gel coating. In some embodiments, the sol precursor solution may include a base catalyst. In some embodiments, the substrate is coated by dip-coating, spread-coating, spray coating or combinations thereof. In some embodiments, the substrate is coated with two or more layers of the sol and/or an additional coating material. In addition, in some embodiments, the co-condensing the sol precursor solution includes co-condensing backbone alkoxysilane in the absence of the diazeniumdiolate-modified alkoxysilane for a specified time, and then adding the diazeniumdiolate-modified alkoxysilane to form the sol.

### BRIEF DESCRIPTION OF THE FIGURES

FIG. 1 provides examples of particular diazeniumdiolate-modified alkoxysilanes that may be used to form coatings in some embodiments of the invention.

FIGS. 2A-2I provide examples of particular multifunctional alkoxysilanes that may be used to form coatings in some embodiments of the invention.

FIG. 3 illustrates a coating on a metallic medical device according to some embodiments of the invention.

FIG. 4 illustrates how backbone alkoxysilanes and/or diazeniumdiolate-modified alkoxysilanes may be bound to a metal surface according to some embodiments of the invention.

FIG. 5 is a graph illustrating NO flux over time for coatings according to some embodiments of the invention.

FIG. 6 provides a graph comparing NO storage of a coating according to an embodiment of the invention (“Pre-Charged”) with two post-charged NO-releasing coatings (“Comparative Example” and “Post-Charged”).

### DETAILED DESCRIPTION OF EMBODIMENTS OF THE INVENTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this invention belongs.

All patents, patent applications and publications referred to herein are incorporated by reference in their entirety. In the event of conflicting terminology, the present specification is controlling.

The embodiments described in one aspect of the present invention are not limited to the aspect described. The embodiments may also be applied to a different aspect of the invention as long as the embodiments do not prevent these aspects of the invention from operating for its intended purpose.

### CHEMICAL DEFINITIONS

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl)hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. Exemplary branched alkyl groups include, but are not limited to, isopropyl, isobutyl, tert-butyl. “Lower alkyl” refers to an alkyl group having 1 to about 8 carbon atoms (i.e., a C₁₋₈ alkyl), e.g., 1, 2, 3, 4, 5, 6, 7, or 8 carbon atoms, “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₅ straight-chain alkyls. In other embodiments, “alkyl” refers, in particular, to C₁₋₅ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, aryloxyl, acylamino, aroylamino, carbamoyl, alkylcarbamoyl, dialkylcarbamoyl, arylthio, alkylthio, alkylene, and —NR¹R″, wherein R¹ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto. Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

“Alkoxyl” refers to an alkyl-O— group wherein alkyl is as previously described. The term “alkoxyl” as used herein can refer to, for example, methoxyl, ethoxyl, propoxyl, isopropoxyl, butoxyl, f-butoxyl, and pentoxyl. The term “oxyalkyl” can be used interchangeably with “alkoxyl”. In some embodiments, the alkoxyl has 1, 2, 3, 4, or 5 carbons.

“Aralkyl” refers to an aryl-alkyl group wherein aryl and alkyl are as previously described, and included substituted aryl and substituted alkyl. Exemplary aralkyl groups include benzyl, phenylethyl, and naphthylmethyl.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Arylene” refers to a bivalent aryl group. An exemplary arylene is phenylene, which can have ring carbon atoms available for bonding in ortho, meta, or para positions with regard to each other, i.e., respectively. The arylene group can also be napthylene. The arylene group can be optionally substituted (a “substituted arylene”) with one or more “aryl group substituents” as defined herein, which can be the same or different.

“Aralkylene” refers to a bivalent group that contains both alkyl and aryl groups. For example, aralkylene groups can have two alkyl groups and an aryl group (i.e., -alkyl-aryl-alkyl-), one alkyl group and one aryl group (i.e., -alkyl-aryl-) or two aryl groups and one alkyl group (i.e., -aryl-alkyl-aryl-).

The term “amino” and “amine” refer to nitrogen-containing groups such as NR₃, NH₃, NHR₂, and NH₂R, wherein R can be alkyl, branched alkyl, cycloalkyl, aryl, alkylene, arylene, aralkylene. Thus, “amino” as used herein can refer to a primary amine, a secondary amine, or a tertiary amine. In some embodiments, one R of an amino group can be a cation stabilized diazeniumdiolate (i.e., NONO⁻X⁺). The terms “cationic amine” and “quaternary amine” refer to an amino group having an additional (i.e., a fourth) R group, for example a hydrogen or an alkyl group bonded to the nitrogen. Thus, cationic and quaternary amines carry a positive charge.

The term “alkylamine” refers to the -alkyl-NH₂ group.

The term “carbonyl” refers to the —(C═O)— group.

The term “carboxyl” refers to the —COOH group and the term “carboxylate” refers to an anion formed from a carboxyl group, i.e., —COO⁻.

The terms “halo”, “halide”, or “halogen” as used herein refer to fluoro, chloro, bromo, and iodo groups.

The term “hydroxyl” and “hydroxy” refer to the —OH group.

The term “hydroxyalkyl” refers to an alkyl group substituted with an —OH group.

The term “mercapto” or “thio” refers to the —SH group. The term “silyl” refers to groups comprising silicon atoms (Si).

The term “silane” refers to any compound that includes four organic groups, such as including any of the organic groups described herein (e.g., alkyl, aryl and alkoxy), bonded to a silicon atom.

As used herein the term “alkoxysilane” refers to a silane that includes one, two, three, or four alkoxy groups bonded to a silicon atom. For example, tetraalkoxysilane refers to Si(OR)₄, wherein R is alkyl. Each alkyl group can be the same or different. An “alkylalkoxylsilane” refers to an alkoxysilane wherein one or more of the alkoxy groups has been replaced with an alkyl group. Thus, an alkylalkoxysilane comprises at least one alkyl-Si bond.

The term “fluorinated silane” refers to an alkylsilane wherein one of the alkyl groups is substituted with one or more fluorine atoms.

The term “cationic or anionic silane” refers to an alkylsilane wherein one of the alkyl groups is further substituted with an alkyl substituent that has a positive (i.e., cationic) or a negative (i.e. anionic) charge, or can become charged (i.e., is ionizable) in a particular environment (i.e., in vivo).

The term “silanol” refers to a Si—OH group.

### NO-Releasing Sol-Gel Coatings

Provided according to some embodiments of the invention are NO-releasing sol-gel coatings that are formed from a sol precursor solution that includes backbone alkoxysilanes and diazeniumdiolate-modified alkoxysilanes. As used herein, the term “backbone alkoxysilane” refers to an alkoxysilane that is not modified with an NO-releasing functional group such as a diazeniumdiolate.

Any suitable backbone alkoxysilane, or mixtures thereof; may be included in the sol precursor solution. However, in some embodiments, the backbone alkoxysilane may include a tetraalkoxysilane having the formula Si(OR)₄, wherein each R is independently an H, alkyl or substituted alkyl. As such, the R groups in the backbone alkoxysilane may be the same or may be different. In particular embodiments, the tetraalkoxysilane may include tetramethoxysilane (TMOS), tetraethoxysilane (TEOS), tetra-n-propoxysilane (TPOS) and/or tetra-n-butoxysilane (TBOS). In some embodiments of the invention, the backbone alkoxysilane may include an alkylalkoxysilane having the formula of R′—Si(OR)₃, wherein R′ is an organic functional group (e.g., alkyl, aryl or alkylaryl) and each R is independently H, alkyl or substituted alkyl. As such, each R may be the same or may be different and each R group may be the same or different as R′. In particular embodiments, the backbone alkoxysilane may include methyltrimethoxysilane (MTMOS), ethyltrimethoxysilane (ETMOS), propyltrimethoxysilane (PTMOS), butyltrimethoxysilane (BTMOS), butyltriethoxysilane (BTEOS), and/or octadecyltrimethoxysilane (ODTMOS). In some embodiments of the invention, the backbone alkoxysilane may include an alkoxysilane having the formula R′R″—Si(OR)₂, wherein R′ and R″ are each independently an organic functional group (e.g., alkyl, aryl or alkylaryl) and each R is independently H, alkyl or substituted alkyl. In some embodiments of the invention, the backbone alkoxysilane may include an alkoxysilane having the formula of R′R″R′″—SiOR, wherein R′, R″ and R′″ are each independently an organic functional group (e.g., alkyl, aryl or alkylaryl) and R is H, alkyl or substituted alkyl.

Examples of backbone alkoxysilanes that may be used in some embodiments of the invention include acryloxypropylmethyldimethoxysilane, 3-acryloxypropyltrimethoxysilane, allyltriethoxysilane, allytrimethoxysilane, amyltriethoxysilane, amyltrimethoxysilane, 5-(bicycloheptenyl)methyltriethoxysilane, 5-(bicycloheptenyl)methyltrimethoxysilane, 5-(bicycloheptenyl)dimethylmethoxysilane, 5-(bicycloheptenyl)methyldiethoxysilane, bis(3-cyanopropyl)diethoxysilane, bis(3-cyanopropyl)dimethoxysilane, 1,6-bis(trimethoxysilyl)hexane, bis(trimethylsiloxy)methylsilane, bromomethyldimethylmethoxysilane, 3-bromopropyltriethoxysilane, n-butyldimethylmethoxysilane, tert-diphenylmethoxysilane, n-butyldimethoxysilane, n-butyldiethoxysilane, n-butyltrimethoxysilane, 2-(carbomethoxy)ethyltrimethoxysilane, 4-chlorobutyldimethylmethoxysilane, 4-chlorobutyldimethylethoxysilane, 2-chloroethyltriethoxysilane, chloromethyldimethylethoxysilane, p-(chloromethyl)phenyltriethoxysilane, p-(chloromethyl)phenyltrimethoxysilane, chloromethyltriethoxysilane, chlorophenyltrimethoxysilane, 3-chloropropylmethyldimethoxysilane, 3-chloropropyltriethoxysilane, 2-cyanoethylmethyltrimethoxysilane, (cyanomethylphenethyl)triethoxysilane, 2-(3-cyclohexenyl)ethyl]trimethoxysilane, cyclohexydiethoxymethylsilane, cyclopentyltrimethoxysilane, di-n-butyldimethoxysilane, dicyclopentyldimethoxysilane, diethyldiethoxysilane, diethyldimethoxysilane, diethyldibutoxysilane, diethylphosphatoethyltriethoxysilane, diethyl(triethoxysilylpropyl)malonate, di-n-hexyldimethoxysilane, diisopropyldimethoxysilane, dimethyldimethoxysilane, 2,3-dimethylpropyldimethylethoxysilane, dimethylethoxysilane, diphenydiethoxysilane, diphenyldimethoxysilane, diphenylmethylethoxysilane, 2-(diphenylphosphino)ethyltriethoxysilane, divinylethoxysilane, n-dodecyltriethoxysilane, epoxycyclohexyl)ethyltrimethoxysilane, ethyltriethoxysilane, ethyltrimethoxysilane, 3-glycidoxypropyldimethylethoxysilane, (3-glycidoxypropyl)methyldimethoxysilane, 3-glycidoxypropyltrimethoxysilane, n-heptylmethyldimethoxysilane, n-hexadecyltriethoxysilane, 5-hexenyltrimethoxysilane, n-hexytriethoxysilane, n-hexyltnethoxysilane, 3-iodopropyltriethoxysilane, 3-iodopropyltrimethoxysilane, isobutyltrimethoxysilane, isobutyltriethoxysilane, isocyanatopropyldimethylmethoxysilane, 3-isocyanatopropyltriethoxysilane, isooctyltriethoxysilane, 3-mercaptopropylmethyldimethoxysilane, 3-mercaptopropyltrimethoxysilane, 3-mercaptopropyltriethoxysilane, 3-methacryloxypropylmethyldiethoxysilane, 3-methacryloxypropyl-methyldiethoxysilane, 3-methacryloxypropyltrimethoxysilane, 3-(4-methoxyphenyl)propyltrimethoxysilane, methylcyclohexyldiethoxysilane, methyldiethoxysilane, methyldimethoxysilane, methyldodecyldiethoxysilane, methyl-n-octadecyldimethoxysilane, methyl(2-phenethyl)dimethoxysilane, methylphenyldiethoxysilane, methylphenyldimethoxysilane, methyl-n-propyldimethoxysilane, methyltriethoxysilane, neophylmethyldiethoxysilane, n-octadecyldimethylmethoxysilane, n-octadecyltriethoxysilane, n-octadecyltrimethoxysilane, octenyltrimethoxysilane, n-octylmethyldimethoxysilane, n-octyltriethoxysilane, phenethyldimethylmethoxysilane, phenethyltriethoxysilane, phenyldimethylethoxysilane, phenyltriethoxysilane, phenyltriethoxysilane, phthalocyanatodimethoxysilane, n-propyltrimethoxysilane, styrylethyltrimethoxysilane, tetra-n-butoxysilane, tetraethoxysilane, tetrapropoxysilane, (tridecafluoro-1,1,2,2,-tetrahydrooctyl)-1-trimethoxysilane, triethoxysilane, triethoxysilylpropylethyl carbamate, triethylethoxysilane, (3,3,3-trifluoropropyl)methyldimethoxysilane, (3,3,3-trifluoropropyl)triethoxysilane, trimethoxysilane, 1-trimethoxysilyl-2-(p,m-chloromethyl)phenylethane, trimethylethoxysilane, 2-(trimethylsiloxy)ethyl methacrylate, p-trimethylsiloxynitrobenzene, triphenylethoxysilane, n-undeceyltriethoxysilane, vinyldimethylethoxysilane and vinyltrimethoxysilane.

The particular backbone alkoxysilanes used and ratio of each in the sol precursor solution may be varied depending on the particular diazeniumdiolate-modified alkoxysilanes present in the sol, the particular substrate coated, the porosity of the coating desired, the hydrophobicity of the coating desired, and the NO-release kinetics desired.

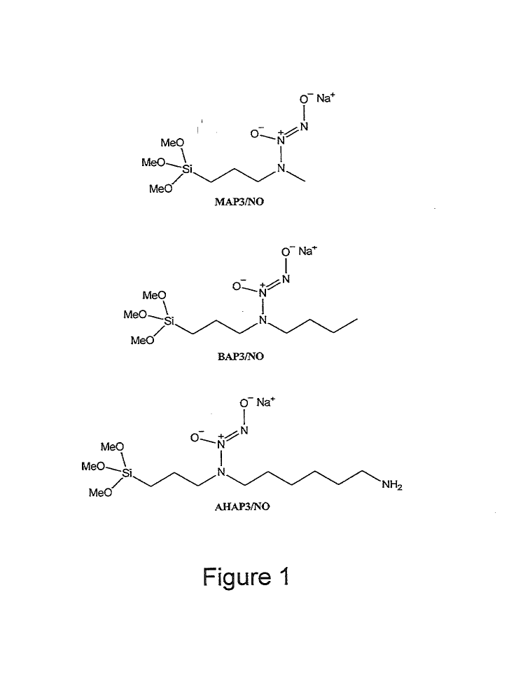

Any suitable diazeniumdiolate-modified alkoxysilane, or mixtures thereof, may be included in the sol precursor solution. In some embodiments of the invention, the diazeniumdiolate-modified alkoxysilane may include a diazeniumdiolate-modified alkoxysilane having the formula of R″—N(NONO⁻X⁺)—R′—Si(OR)₃, wherein each R is independently H, alkyl or substituted alkyl; R′ is substituted or unsubstituted alkylene, substituted or unsubstituted arylene, substituted or unsubstituted alkylarylene or substituted or unsubstituted arylalkylene; R″ is H, alkyl or substituted alkyl; and X⁺ is a monovalent cation such as Na⁺, K⁺, Cs⁺, or Li⁺, a divalent cation, or a cationic amine. Examples of particular diazeniumdiolate-modified alkoxysilanes are shown in FIG. 1. The diazeniumdiolate-modified alkoxysilane may prepared by any suitable method. However, methods of synthesizing diazeniumdiolate-modified alkoxysilanes are described in U.S. Patent Application Publication No, 2009/0214618 to Schoenfisch et al., which is hereby incorporated by reference herein in its entirety.

As an example, a diazeniumdiolate-modified alkoxysilane may be prepared by exposing an appropriate aminoalkoxysilane to NO gas (e.g., between 1 and 34 atm) in a solution, such as a solution that includes sodium methoxide and a methanol co-solvent. In some embodiments the ratio of sodium methoxide to aminoalkoxysilane ranges from 0.8:1 to 1.25:1 to maximize the conversion of the amines to diazeniumdiolate NO donors. In such cases, any suitable aminoalkoxysilane may be used. However, in some embodiments, the aminoalkoxysilane may include a primary amine such as 3-aminopropyltrimethoxysilane (APTMS); 3-aminopropyltriethoxysilane (APTES); 4-aminobutyltriethoxysilane (ABTES); 4-amino-3,3-dimethylbutyltrimethoxysilane (ADBTMS); a secondary amine such as [3-(methylamino)propyl]trimethoxysilane (MAP3); N-butylamino-propyltrimethoxysilane(n-BAP3); t-butylamino-propyltrimethoxysilane(t-BAP3); 3-(N-styrylmethyl-2-aminoethylamino)propyltrimethoxysilane (SEAP3); N-ethylaminoisobutyltrimethoxysilane(EAiB3); N-phenylamino-propyltrimethoxysilane (PAP3); and N-cyclohexylaminomethyltrimethoxysilane (cHAM3); N-cyclohexylaminopropyltrimethoxysilane (cHAP3); diamines such as (aminoethylaminomethyl)phenethyltrimethoxysilane (AEMP3); N-(6-aminohexyl)aminopropyltrimethoxysilane (AHAP3); N-(6-aminohexyl)aminomethyltriethoxysilane) (AHAM3); N-(2-aminoethyl)-3-aminopropyltrimethoxysilane (AEAP3); N-(2-aminoethyl)-11-aminoundecyltrimethoxysilane (AEAUD3); (2-N-benyzlaminoethyl)-3-aminopropyltrimethoxysilane (BEAP3); and/or polyamines such as (3-trimethoxysilylpropyl)diethylenetriamine (DET3). Other aminoalkoxysilanes that may be used in some embodiments of the invention include 3-aminopropyldimethoxysilane, N-(3-acryloxy-2-hydroxypropyl)-3-aminopropyltriethoxysilane, N-2-(aminoethyl)-3-aminopropyltris(2-ethyl-hexyloxy)silane, 3-(m-aminophenoxy)propyltrimethoxysilane, 3-(1-aminopropoxy)-3,3-dimethyl-1-propenyltrimethyoxysilane, 3-aminopropyltris(methoxyethoxyethoxy)silane, 3-aminopropylmethyldiethoxysilane, 3-aminopropyltris(trimethylsiloxy)silane, bis(dimethylamino)methylmethoxysilane, bis(dimethylamino)phenylethoxysilane, bis(2-hydroxyethyl)-3-aminopropyltriethoxysilane, bis(2-hydroxyethyl)-3-aminopropyltrimethoxysilane, bis(3-triethoxysilyl)propylamine, 1,4-bis[3-(trimethoxysilyl)propyl]ethylenediamine, (N,N-diethyl-3-aminopropyl)trimethoxysilane, (N,N-dimethyl-3-aminopropyl)trimethoxysilane, N-phenylaminopropyltrimethoxysilane, trimethoxysilylpropyldiethylenetriamine, trimethoxysilylpropylpentaethylenehexamine, triethoxysilyloctyldiethylenetriamine, triisopropoxysilylpentaethylenehexamine, 3-aminopropylmethyldiethoxysilane, 2-(perfluorooetyl)ethyltriaminotrimethoxysilane, 4-aminobutyltrimethoxysilane, N-(6-aminohexyl)aminopropyltrimethoxysilane, 3-(dimethoxymethylsilylpropyl)diethylenetriamine, N-(2-aminoethyl)-N-[3-(dimethoxymethylsilyl)propyl]-1,2-ethanediamine and amine-modified polydimethylsiloxane copolymer (available from Dow Corning as “MDX4-4159”).

In some embodiments, the aminoalkoxysilane may have the formula: NH[R′—Si(OR)₃]₂, wherein R is H, alkyl or substituted alkyl and R′ is substituted or unsubstituted alkylene, substituted or unsubstituted arylene, substituted or unsubstituted alkylarylene or substituted or unsubstituted arylalkylene. In some embodiments, the diazeniumdiolate modified alkoxysilane may include a dipodal aminoalkoxysilane such as bis-(trimethoxysilylpropyl)amine, bis(triethoxysilylpropyl)amine, bis-(triethoxysilylpropyl)ethylene diamine, N-[2-vinylbenzylamino)ethyl]-3-aminopropyltrimethoxysilane, aminoethylaminopropyltrimethoxysilane, trimethoxysilyl-modified polyethylenimine, methyldimethoxysilyl-modified polyethylenimine, bis-[(3-trimethoxysilyppropyl]ethylenediamine, bis(methyldiethoxysilylpropyl)amine, bis(triethoxysilylmethyl)amine, N,N′-bis[(3-trimethoxysilyl)propyl]ethylenediamine.

In some embodiments of the invention, the diazeniumdiolate-modified alkoxysilane may be O²-protected prior to the preparation of sol-gel coatings. Such O²-protected diazeniumdiolate modified aminoalkoxysilanes may have the formula: R″—N(NONO—R′″)—R′—Si(OR)₃, wherein each R is independently H, alkyl or substituted alkyl, R′ is substituted or unsubstituted alkylene, substituted or unsubstituted arylene, substituted or unsubstituted alkylarylene or substituted or unsubstituted arylalkylene, R″ is H, alkyl or substituted alkyl and R′″ is a protecting group that imparts pH dependent, enzymatic, photolytic, or thiolation triggering mechanisms. Such protecting groups are known to those skilled in the art of forming O²-protected diazeniumdiolates.

In some embodiments of the invention, the sol precursor solution may include at least one multifunctional alkoxysilane. The term “multifunctional alkoxysilane” refers to an alkoxysilane that includes at least one functionality that provides at least one additional property to the sol-gel coating. The multifunctional alkoxysilane may be a backbone alkoxysilane, a diazeniumdiolate-modified alkoxysilane or may be a different alkoxysilane. In some embodiments, the multifunctional alkoxysilane has the formula R′R″R′″SiOR, wherein R is H, alkyl or substituted alkyl, R′, R″ and R′″ are each independently a substituted or unsubstituted alkyl, a substituted or unsubstituted aryl, a substituted or unsubstituted alkylaryl, a substituted or unsubstituted arylalkyl or an organic moiety that provides at least one additional property to the sol-gel coating. At least one of R′, R″ and R′″ is an organic moiety necessary that provides the at least one additional property to the sol-gel coating. This organic moiety may be chosen based on the property desired and the stability of a particular functionality under sol-gel processing conditions. The multifunctional alkoxysilane may be introduced into the sol precursor solution with the backbone alkoxysilane and diazeniumdiolate-modified alkoxysilane to form a sol which may then form a multifunctional co-condensed siloxane coating. Examples of additional properties that may be imparted to a substrate via the multifunctional alkoxysilane include:

Anti-Corrosive

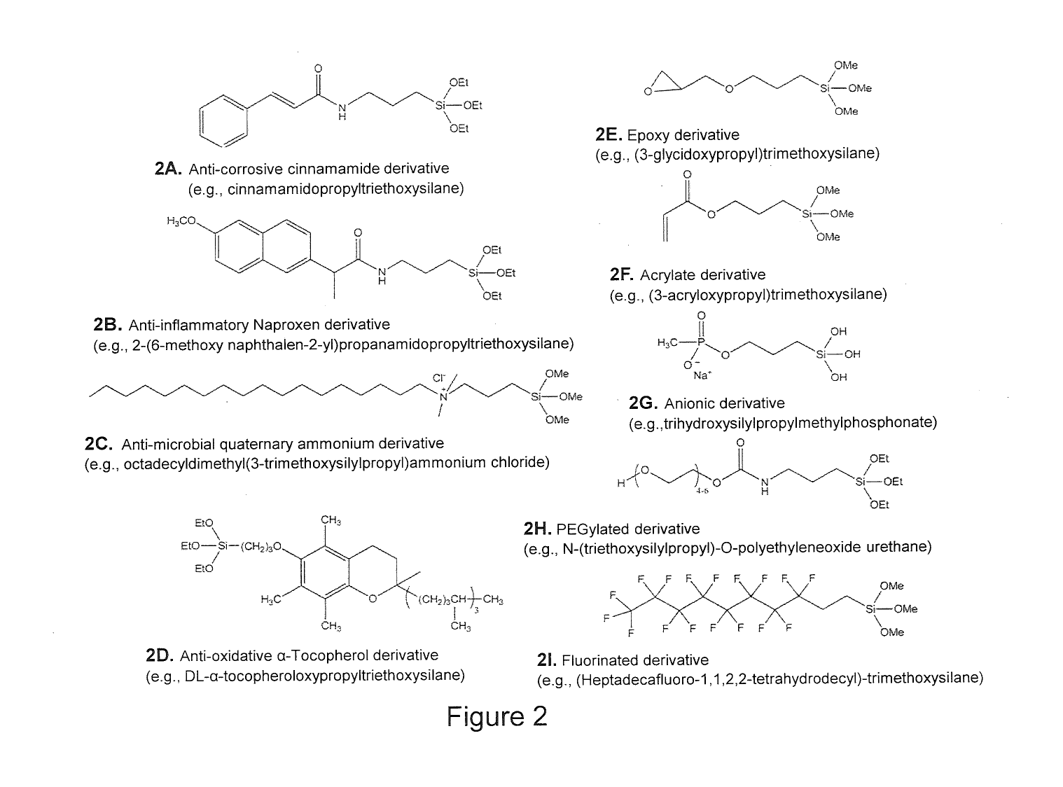

Any suitable alkoxysilane that may impart anti-corrosive properties to the sol-gel coating may be used. Common inhibitors known to one skilled in the art to prevent corrosion of metallic surfaces include imines formed from the condensation products of aldehydes and amines, cinnamaldehyde and ascorbic acid. As such, in some embodiments, the multifunctional alkoxysilane may include a dipodal alkoxysilane formed from the condensation of glutaraldehyde and two 3-aminopropyltrimethoxysilanes and/or the cinnamamide silane derivative shown FIG. 2A.

Anti-Inflammatory

Any suitable alkoxysilane that may impart anti-inflammatory properties to the sol-gel coating may be used. Widely accepted anti-inflammatory agents including ibuprofen, diclofenac, and naproxen may be covalently attached to a medical device surface to minimize inflammation and pain caused by device implantation. As such, in some embodiments, the multifunctional alkoxysilane may include an ibuprofen alkoxysilane derivative, a diclofenac alkoxysilane derivative or a naproxen alkoxysilane derivative, such as that shown in FIG. 2B. Ester linkages sensitive to enzymatic or hydrolytic cleavage may also be employed to provide controlled release of the anti-inflammatory agent into the surrounding tissue.

Anti-Microbial

Any suitable alkoxysilane that may impart antimicrobial properties to the sol-gel coating may be used. Broad spectrum antimicrobial agents including quaternary ammonium compounds, chlorhexidine, polyhexamethylene biguanide, triclosan, ionic silver complexes, iodine, and hypochlorite may be derivatized with an alkoxysilane to provide microbicidal activity to the device surface or the surrounding tissue. In some embodiments, the multifunctional alkoxysilane may include a quaternary ammonium alkoxysilane derivative, a chlorhexidene alkoxysilane derivative, a polyhexamethylene biguanide alkoxysilane derivative, a triclosan alkoxysilane derivative, an ionic silver alkoxysilane derivative, a iodine-releasing alkoxysilane derivative and a hypochlorite alkoxysilane derivative. In particular embodiments, the quaternary ammonium derivative may be the octadecyldimethyl(3-trimethoxysilylpropyl)ammonium chloride shown in FIG. 2C.

Anti-Oxidative

Any suitable alkoxysilane that may impart anti-oxidative properties to the sol-gel coating may be used. In some embodiments, the multifunctional alkoxysilane may include a vitamin E alkoxysilane derivative, an ascorbic acid alkoxysilane derivative, a glutathione alkoxysilane derivative, a N-acetylcysteine alkoxysilane derivative and other thiol alkoxysilane derivatives. Such alkoxysilanes may be incorporated into medical device coatings to mediate oxidative stress at the implant surface or in the surrounding tissue. In particular embodiments, the multifunctional alkoxysilane includes the DL-αtocopheroloxypropyltriethoxyalkoxysilane shown in FIG. 2D.

Crosslinking

Any suitable alkoxysilane that may impart additional crosslinking to the sol-gel coating may be used. Functional alkoxysilanes are routinely used by those skilled in the art of sol-gel chemistry to enable methods of curing and forming a stable siloxane network via covalent bonding other than siloxane bonds. In the present invention this affords a method for forming stable diazeniumdiolated aminoalkoxysilane coatings that do not involve sintering at high temperatures that may decompose the pre-loaded NO donors. In some embodiments, the multifunctional alkoxysilane may include an epoxy group including (3-glycidoxypropyl)trimethoxysilane (shown in FIG. 2E), (3-glycidoxypropyltriethoxysilane), (3-glycidoxypropyl)methyldiethoxysilane), 1,3-bis(glycidoxypropyl)tetramethyl-disiloxane, epoxycyclohexyl)ethyltrimethoxysilane; acrylo including (3-acryloxypropyl)trimethoxysilane (shown in FIG. 2F), acryloxymethyltrirnethoxysilane, methacryloxypropyltriethoxysilane, methacryloxypropyltrimethoxysilane); isocyano including 3-isocyanatopropyltriethoxysilane, isocyanatopropyltrimethoxysilane; vinyl including vinylmethyldiethoxysilane, vinylmethyldimethoxysilane amino, vinyltriethoxysilane, vinyltrimethoxysilane, vinyltriisopropxysilane; and amino including 3-aminopropyltrimethoxysilane, 3-aminopropyltriethoxysilane, 4-aminobutyltriethoxysilane. In addition to intra-silane bonding, the functionalities may be used to facilitate crosslinking of the sol-gel coating with a top coated polymer layer. The top-coated polymer layer may be polymerized at the surface and react with the R₃ functionality from the multifunctional alkoxysilane in the sol-gel coating in order to facilitate bonding between the two surface layers and prevent delamination of the polymer top-coat. The top-coat may be applied during device fabrication or applied on a macroscopic scale upon device implantation as is the case with methacrylate-based bone cement used to anchor artificial joints. Acrylate or methacrylate derivatized alkoxysilane residues can participate in the free-radical initiated polymerization of the two bone cement monomers.

Surface Charge

Any suitable alkoxysilane that may impart surface charge to the sol-gel coating may be used. One of the most widely known strategies to alter protein adsorption, bacterial adhesion, and concomitant biofouling of implantable devices is to alter the charge of the implant surface. However, these passive surface functionalities alone have been unable to dramatically improve foreign body response. In the present invention, the combination of nitric oxide and surface charge may provide medical devices with improved biocompatibility. Thus, in some embodiments, the multifunctional alkoxysilane may include a cationic alkoxysilane such as (2-N-benyzlaminoethyl)-3-aminopropyl-trimethoxysilane, hydrocholoride; bis(methoxyethyl)-3-trimethoxysilylpropyl-ammonium chloride; N—N-didecyl-N-methyl-N-(3-trimethoxysilyl)ammonium chloride; N-trimethyoxysilylpropyl-N,N,N-trimethyl ammonium chloride; octadecylbis(triethoxysilylpropyl)-ammonium chloride; and octadecyldimethyl(3-trimethoxysilylpropyl)ammonium chloride. In some embodiments, the multifunctional alkoxysilane may include an anionic alkoxysilanes such as 3-trihydroxysilylpropylmethyl phosphonate, sodium salt (shown in FIG. 2G) and carboxyethylsilanetriol, sodium salt.

Surface Hydrophilicity

Any suitable alkoxysilane that may impart hydrophilic properties to the sol-gel coating may be used. Alkoxysilanes containing repeat poly(ethylene)oxy groups may be used to increase the wettability of the NO-releasing coating thereby helping to improve biocompatibility upon implantation and also enhance the rate of water uptake in the co-condensed siloxane coating. Surface hydrophilicity can thus be utilized to enhance the NO-release kinetics of the diazeniumdiolated aminoalkoxysilane derivatives. Therefore, in some embodiments, the multifunctional alkoxysilane may include a hydrophilic silane such as N-triethoxysilylpropyl)-O-polyethyleneoxide urethane (shown in FIG. 2H); N-3-[amino(polypropylenoxy)]aminopropyltrimethoxysilane; bis-[3-(triethoxysilylpropoxy)-2-hydroxypropoxy]polyethylene oxide; bis(3-triethoxysilylpropyl)polyethylene oxide (25-30); [hydroxy(polyethyleneoxy)propyl]-triethoxysilane; and 2-[methoxy(polyethyleneoxy)propyl]-trimethoxysilane.

Surface Hydrophobicity

Any suitable alkoxysilane that may impart hydrophobic properties to the sol-gel coating may be used. Hydrophobic silanes are known to those skilled in the art to increase lipophilicity of surfaces. In some embodiments, the multifunctional alkoxysilane may include linear alkyl, branched and cyclic alkylalkoxysilanes having at least three carbon atoms, substituted and unsubstituted phenyl alkoxysilanes, and fluorinated alkoxysilanes. A surprising discovery of the current invention is that diazeniumdiolated aminoalkoxysilane networks exhibit excellent coating stability and uniformity when a suitable amount of fluoroalkoxysilane is added to the sol precursor solution. For example, a concentration of 10-20% (v/v) fluoroalkoxysilane may be included in the sol precursor solution. Exemplary fluoroalkoxysilanes may include heptadecafluoro-1,1,2-2-tetrahydrodecyl)triethoxysilane (shown in FIG. 2I), (3,3,3-trifluoropropyl)trimethoxysilane, (perfluoroalkyl)ethyltriethoxysilane, nonafluorohexyltrimethoxysilane, nonafluorohexyltriethoxysilane, (tridecafluoro-1,1,2,2-tetrahydrooctyl)triethoxysilane, and (tridecafluoro-1,1,2,2-tetrahydrooctyl)trimethoxysilane.

The silane precursors may be combined in any suitable ratio in the sol. In some embodiments, the silane precursor solution includes a backbone alkoxysilane at a concentration in a range of about 1 to about 99 percent by volume and a diazeniumdiolate-functional aminoalkoxysilane at a concentration in a range of about 1 to about 99 percent by volume. In particular embodiments, the concentration of the diazeniumdiolate-functional aminoalkoxysilane may be in a range of about 1 to about 40 percent by volume, and in some embodiments, 10 to 40% by volume, and the concentration of the backbone alkoxysilane may be in a range of about 60 to about 90 percent by volume.

Percentages of each silane precursor in the sol precursor solution may be varied to affect the NO-release amount and rate, porosity of the xerogel matrix, thickness of the coating, stability and coating integrity, and contribution of additional functionality to the coating. For example, if there is not enough backbone alkoxysilane in the sol, a siloxane network may not form, resulting in an amorphous gel. Furthermore, the total silane concentration in the sol precursor solution may affect the thickness and stability of the film. In some embodiments, the total silane concentration is in a range of about 1 to about 5 mM, and in some embodiments, in a range of about 1 to 3 mM, and in some embodiments about 2 mM.

The volume and type of the solvent employed in the sol precursor solution may vary. Examples of solvents include water, methanol, ethanol, propanol, butanol, 2-ethoxyethanol, formamide, dimethylformamide, dioxane, tetrahydrofuran, and mixtures thereof. In some embodiments, drying control additives may be included in the sol to facilitate the drying of the gels. Such drying control additives may allow for drying of the gel without cracking. Examples of drying control additives include formamide, dimethylformamide, diethylamine amine, acetonitrile, dioxane, glycerol, oxalic acid, surfactants and mixtures thereof.

In some embodiments of the invention, the sol precursor solution may include a base catalyst. The base catalyst may initiate the sol-gel process for making diazeniumdiolate-functional aminoalkoxysilane coatings. Any suitable base catalyst may be used. However, examples of base catalysts include ammonia, alkali metal hydroxides, fluorides (NaF) and organic bases. In some embodiments, the concentration of the base catalyst is in a range of about 0.1 to about 10% v/v of the sol solution (0.5 mM-50 mM). Acid catalysts in aqueous or alcoholic solutions at neutral or acidic pH used to form previous aminoalkoxysilane xerogel coatings requiring post-loading of NO lead to spontaneous diazeniumdiolate decomposition and loss of NO donor functionality. However, in other embodiments that contain O²-protected diazeniumdiolate-modified alkoxysilanes with enhanced stability, acid catalysts may be employed.

In some embodiments of the invention, a radical initiator may be added to the sol precursor solution. Any suitable radical initiator may be used, but in some embodiments, initiators may include organic peroxides and azo compounds (e.g. azobisisobutyronitrile, AIBN) that may be used to initiate polymerization of modified alkoxysilanes (e.g. 3-methacryloxypropyl trimethoxysilane) to strengthen the siloxane coating.

In some embodiments of the invention, a porogen may be included in the sol precursor solution. Control of porosity of the sol may enable increased or decreased water uptake of the coating, and thus, may allow for control of the proton initiated decomposition of the diazeniumdiolate modified aminoalkoxysilanes, may facilitate tissue and bone ingrowth on and into the device, and may provide a mechanism for analyte diffusion in the case of sensor-based implants. Any suitable porogen may be used. However, examples of porogens include dendrimers, water soluble polymers such as PVP, PVA, PEG, and biodegradable polymers such as PLA, PGA, PLGA, caprolactones, polyesters and polypeptides. In some embodiments, the concentration of the porogen may be in a range of from about 0.05 to about 20% (w/v) of the cast sol solution. The molecular weights and resulting macromolecular structure of the sol may govern pore size and geometry.

The particular procedure used to form the sol from the sol precursor solution may vary based on the identity of aminoalkoxysilane used to form the diazeniumdiolate-modified alkoxysilane because the rate of hydrolysis and condensation reactions in the sol may be dependent on the type of amine linkage and organic character of the alkoxy substituents. For diazeniumdiolate-modified alkoxysilanes that have a relatively fast rate of hydrolysis and condensation, a shorter sol mixing time may be desirable. For diazeniumdiolate-modified alkoxysilanes that have a relatively slow rate of hydrolysis and condensation, a longer mixing time may be needed. It has been surprisingly discovered that, in some embodiments, in order for suitable coatings to form, the rate of hydrolysis of the backbone alkoxysilane and the diazeniumdiolated aminoalkoxysilane should be on the same time scale ranging from seconds, to minutes, to hours. The stability of the alkoxide leaving groups in solution, pH of the sol precursor solution, and concentration of catalyst all affect the hydrolysis and subsequent co-condensation rates. Further, any additional alkoxysilanes added to the precursor solution may also affect the rates of hydrolysis and condensation in the sol by altering polarity, disrupting hydrogen bonding, and enhancing/decreasing siloxane oligomer solubility.

The order and rate of addition of particular reagents may affect the properties of the resulting NO-releasing sol-gel coating. For example, the sol may be prepared in one step, or in two or more steps. In a two step process, in some embodiments, the backbone alkoxysilane may be allowed to react first, and then the diazeniumdiolate-modified alkoxysilane may be added later. For example, in some embodiments, the backbone alkoxysilane may react for about one hour prior to addition of the diazeniumdiolate-modified alkoxysilane.

The casting volume may also affect the properties of the coating because it may affect drying time. In some embodiments, the casting volumes may be in the range of from about 1 to about 200 μL/cm², and in particular embodiments, in a range of about 4 to about 30 μL/cm².

Also provided according to some embodiments of the invention are methods of producing NO-releasing sol-gel coatings that include (a) co-condensing a sol precursor solution comprising a backbone alkoxysilane and a diazeniumdiolate-modified alkoxysilane in a solvent to form a sol; (b) coating a substrate with the sol; and (c) drying the sol to form the NO-releasing sol-gel coating. The sol precursor solution may further include any of the components described herein such as a multifunctional alkoxysilane, base catalyst, porogen and free radical initiator, and/or any other additives known in the art of forming sol-gel coatings. Additionally, such methods may be performed by any method known to those of skill in the art.

The substrate may be coated with the sol and/or sol precursor solution to form the coating. In some embodiments of the present invention, methods of coating the substrate include applying the coating to a device via dip-coating, spread-coating, spray coating, spin coating, brushing, imbibing, rolling and/or electrodeposition. Other methods may be used and are known to those of skill in the art.

In some embodiments of the invention, the coating may be applied to the substrate as only one layer. In some embodiments, the substrate may be coated two or more times to a form multi-layered coating. A multi-layered coating may include multiple layers of a single sol-gel containing one NO donor composition according some embodiments of the invention. The multiple layers may allow the combination of relatively thin layers, which may dry more evenly and therefore show less cracking, to form a thicker coated layer. Such a composition may also provide for a coating capable of extended release of NO upon implantation.

Alternatively, a multi-layered coating may include at least one layer formed from a different sol-gel composition according to an embodiment of the invention. Such a combination of different types of NO-releasing sol-gel coatings may impart additional functionality to the device surface. Furthermore, in some embodiments, a multi-layer coating may include at least one coating layer that is formed from a different sol-gel composition or a different type of coating material altogether. For example, a NO-releasing sol-gel coating according to an embodiment of the invention may be top coated with additional polymeric materials that may impart stability to the underlying sol-gel coating and regulate diffusion of water to the diazeniumdiolate functional groups, thus controlling NO-release. Such coatings may also reduce or eliminate biofouling at the surface. Any suitable top coating may be used. However, examples of top coatings include polyurethane, collagen, silicone rubber, polystyrene, polymethylmethacrylate, polyvinylchloride and combinations thereof. While a top coat may be applied, in some embodiments, a NO-releasing sol-gel coating according to an embodiment of the invention is the top layer of a multiple layered coating. As such, a NO-releasing sol-gel coating according to an embodiment of the invention may directly contact an organ or tissue.

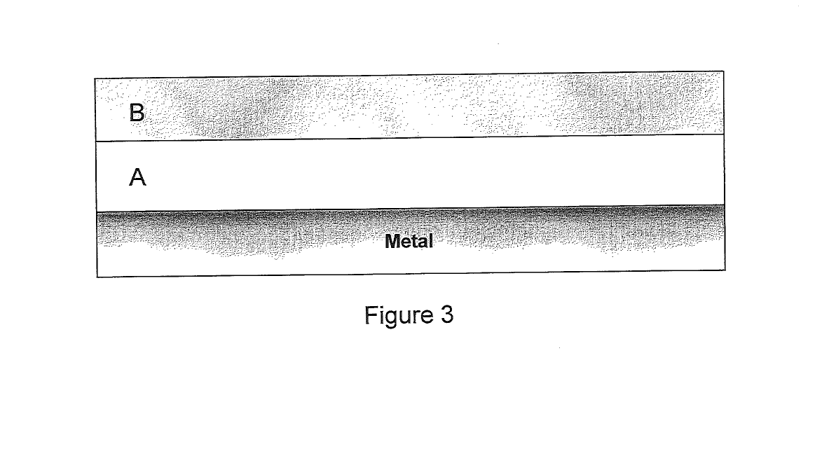

In some embodiments, the surface may be coated with an additional polymer substrate designed to impart passive surface functionality in combination with the NO-released from the underlying sol-gel coating. Examples may include polyurethane, collagen, silicone rubber, polystyrene, polymethylmethacrylate and polyvinylchloride. FIG. 3 illustrates that in some embodiments, a metallic medical device may be coated with (A) a NO-releasing sol-gel coating according to an embodiment of an invention; and (B) a topcoat polymer or additional organosilane layer thereon. In some embodiments, a multifunctional alkoxysilane is included in the sol precursor solution that provides a surface of the a NO-releasing sol-gel coating that allows for the topcoat or additional organosilane layer to remain stably adhered thereto so that delamination of the topcoat does not occur.

According to some embodiments of the invention, provided is a bioactive glass layer in, under or on the sol-gel coating that contains a certain mol % diazeniumdiolate aminoalkoxysilane to impart NO-release. Any suitable bioactive glass may be used, but in some embodiments, the bioactive glass may be 58S, which is 58 wt. % SiO₂—33 wt. % CaO—9 wt. % P₂O₅, which may be modified with a NO-releasing alkoxysilane. For example a 58S composition may be modified with BAP3/NO to include 53 wt, % SiO₂—32 wt, % CaO—9 wt. % P₂O₅—5 wt. % BAP3/NO. Most bioactive glass materials are hydrolyzed in the presence of 2M HNO₃, and as such, when an NO-releasing molecule that is not acid labile is used, the hydrolysis may be performed in the usual manner. However, when the bioactive glass is modified with a diazeniumdiolate, the hydrolysis should proceed with a basic catalyst, such as ammonia. Typically, the bioactive glass is pre-soaked in a solution of Tris buffer complemented with 2.5 mM Si and electrolyte concentrations typical for plasma. The bioactive glass may form an apatite layer upon implantation. The resulting apatite surface functionality may support osteointegration while simultaneously releasing NO to prevent infection and decrease inflammation.

In some embodiments, a sol and/or sol precursor solution according to an embodiment of the invention may be further treated after being applied to the substrate. For example, the coating may be dried under vacuum, photocured, or heat cured to form the sol-gel coating. As additional examples, drying agents may also be applied to aid in the complete co-condensation of the components of the sol precursor solution and to prevent cracking/breaking during evaporation of the sol solvent(s). Additionally the siloxane network may be further aged (i.e., driven to complete conversion of silanols into siloxanes bridges) by exposing the coating and substrate to basic solutions up to several orders of magnitude higher in base concentration than that employed during the coating preparation. In another embodiment, radical initiated polymerization and/or photopolymerization of the coating may be performed to strengthen the siloxane coating.

Coatings according to embodiments of the invention may be of any suitable thickness. The thickness may depend on the number of layers contained within the coating and on the method used to apply the coating. In some embodiments, the total thickness of the coating (including all layers, both NO-releasing co-condensed siloxane coating layers and other layers) may be in a range of from about 0.1 μm to about 1 mm. In particular embodiments, the total thickness of the coating is in a range of about 1 to about 250 μm, and in some embodiments, in a range of about 20 to about 150 μm.

The NO-releasing sol-gel coatings may have desirable properties such as increased NO storage, lengthened NO-release durations, and environmentally triggered mechanisms of NO donor decomposition. Further more, in some embodiments of the invention, the NO-releasing sol-gel coatings may have a total NO storage ranging from about 0.01 to about 10 μmol NO·cm⁻².

### Substrates

NO-releasing sol-gel coatings according to embodiments of the invention may be applied to any suitable substrate. However, in some embodiments, the NO-releasing sol-gel coating may be applied to a medical device. As used herein, the term “medical device” refers to any devices or structures used in medical diagnosis, therapy or surgical procedure, including any physical object that can be implanted into the body or which comes in direct contact with the body. These devices may be useful for diagnostic or therapeutic purposes, and can be implanted for use on a permanent or temporary basis. They may be made to replace and act as a missing biological structure. They may be sensors or probes. They may be devices, such as drug delivery devices, for example, in the form of implantable pills or drug-eluting implants. Medical devices may contain electronics, such as artificial pacemakers, retinal implants, cochlear implants, and pulse-generators. Also included are components of these devices, such as electrical leads and guide wires.

Specific medical devices include but are not limited to orthopedic implants, including replacement joints, re-constructive prosthesis (e.g. maxillofacial prostheses), bone cement, bone defect fillers, spinal cages, bone anchors, bone screws, fracture-fixation plates, screws, and tacks, artificial tendons and ligaments, and dental implants; cardiovascular implants, including vascular grafts, vascular access devices and ports, stents, balloons, pacemakers, myocardial plugs, lead coatings including coatings for pacemaker leads, defibrillation leads and coils; ventricular-assist-device devices (e.g. left ventricular assist hearts and pumps, total artificial hearts, shunts, valves including heart valves and vascular valves, anastomosis clips and rings, suture anchors, tissue staples and ligating clips at surgical sites); ophthalmic implants, including corneal implants, retinal implants, and introcular lenses; drug delivery systems; cochlear implants; tissue screws and tacks; tissue adhesives and sealants; tissue staples and ligating clips at surgical sites; matrices for cell encapsulation and tissue engineering; tissue bulking devices and agents; tissue engineering scaffolds for cartilage, bone, skin and other in vivo tissue regeneration; sutures; suture anchors; surgical drapes; gauze; protective platings; breast enlargement prostheses; ostomy devices and long-term urinary devices; bracheotherapy devices; ventriculo-peritoneal shunts; pumps (including implantable infusion pumps); stents (e.g. coronary vascular stents, arterial stents, peripheral vascular stents, cerebral, urethral, ureteral, biliary, tracheal, gastrointestinal and esophageal stents); stent grafts; catheters (e.g., renal or vascular catheters such as balloon catheters, dialysis catheters, long term tunneled central venous catheters, peripheral venous catheters, short-term central venous catheters, arterial catheters, pulmonary artery Swan-Ganz catheters, urinary catheters, long term non-tunneled central venous catheters, peritoneal catheters, and ventricular catheters); guide wires; trocar needles; electrical leads, balloons; implantable stimulators; implantable pulse generators; filters (e.g., vena cava filters and mesh filters for distil protection devices); vascular grafts, abdominal aortic aneurysm devices such as stents and grafts; dialysis ports, embolization devices including cerebral aneurysm filler coils (including Guglilmi detachable coils and metal coils); embolic agents; bulking agents; septal defect closure devices; anastomosis clips and rings; cannulae; contraceptive intrauterine devices; metal wire ligatures; urethral slings; hernia “meshes;” sensors, including biosensors, and biopsy devices, as well as any other device that is implanted or inserted into the body for medical purposes.

The medical device itself may be formed from or include any suitable material. The material comprising a given medical device is chosen based in part on the particular application; for example, the mechanical properties of the device may need to conform to the natural tissue surrounding it. Thus, a different material may be used, for example, for a sensor versus a suture, and for an orthopedic implant versus a retinal implant. For a discussion of the many materials that can be used in medical devices, see Helmus et al., Toxicologic Pathology 36:70-80 (2008), incorporated herein by reference. Examples of materials that may form or be included in the medical device include metals (including germanium, cobalt, chromium, nickel, aluminum, zirconium, tin, hafnium, vanadium, and titanium), metal alloys (including titanium-niobium, titanium-aluminum-vanadium, titanium-aluminum-niobium, vanadium steel, cobalt chrome, the superalloy CoCrMo, and stainless steel), carbon, carbon fibers, carbon polymer, ceramics and glasses (including oxides, carbides, nitrides, or nitro-carbides of silicon, titanium, tantalum, tungsten; zirconium, niobium, chromium, or aluminum), ceramic-metal composites; synthetic and natural polymers and copolymers (including rubber, nylon, silicone, polyurethane, polyethylene, polyvinyl chloride, polystyrene, polyetheretherketone, polytetrafluoroethylene tetraphthalate, polyethylene tetraphthalate, polytetrafluoroethylene, polyglycolic acid, latex, polyglycolic acid, polylactide-co-glycolide, polylactic acid polymethyl methacrylate; latex, gelatin, collagen, albumin, and globulin) and any combination thereof.

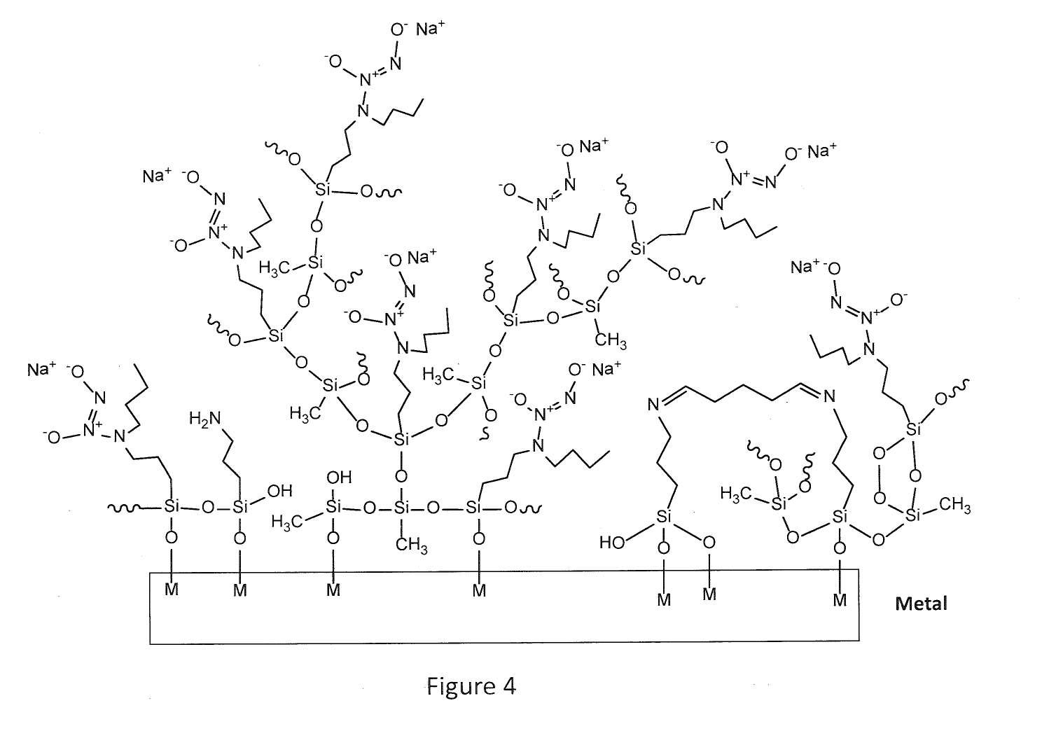

In some embodiments of the present invention, at least one material of a medical device may be pretreated prior to the coating of the device with a NO-releasing sol-gel coating according to an embodiment of the invention. For example, mechanical surface modifications may include machining, grinding, polishing, or blasting metal surfaces prior to deposition of the NO-releasing coating to increase interfacial surface area and allow for increased silane bonding/functionalization. Chemical methods of surface preparation may include alkaline treatment, acidic treatment, hydrogen peroxide treatment, argon and oxygen plasma cleaning, and ozone cleaning. In some embodiments of the present invention, the surface is pretreated with a dipodal alkoxysilane or an aminoalkoxysilane/glutaraldehyde treatment as described in Example 2 to facilitate proper adhesion of the NO-releasing siloxane coating and prevent hydrolysis at the substrate surface. In some embodiments, a metal surface may be pretreated, for example with an alkaline treatment, in order to form a metal hydroxide layer that may react with a silane such as a backbone alkoxysilane, a diazeniumdiolate-modified alkoxysilane. The bonding between the silanes in the sol precursor solution with the metal surface may facilitate adhesion and stability of the sol-gel coating on the surface. FIG. 4 illustrates how backbone alkoxysilane and/or a diazeniumdiolate-modified alkoxysilane may be bound to a metal surface according to some embodiments of the invention. Such binding to hydroxyl moeities on a surface may also be achieved with other surfaces such as glass.

### EXAMPLES

### Example 1

### Precharging Aminosilanes

Sodium methoxide (325 mg) was dissolved in ethanol (3 mL; absolute) and methanol (0.75 mL) via sonication for 5 min. Butylamino-propyltrimethoxysilane (nBAP-3) (1.185 mL) was added and vortexed 1 min to mix. The mixture was divided among two 6 mL glass vials equipped with stir bars, which were then placed in a Parr hydrogenation bomb and affixed to a NO charging apparatus. While stirring, the Parr hydrogenation bomb was flushed with 5 atm of argon three times in rapid succession and then 3 times for 10 minutes each. The bomb was then pressurized with 5 atm of NO (99.5%; further purified over potassium hydroxide for >3 hr) for 3 days to modify the secondary amines to diazeniumdiolates. Following NO modification, the bomb was flushed thrice quickly with argon (5 atm). The resulting BAP-3/NO solution was used immediately.

### Example 2

### Glass Slide Pre-Treatment

Glass slides (9×12.5 mm) were cleaned by sonication in ethanol (absolute) for 20 minutes each. The slides were then gently dried with a stream of nitrogen and then soaked in 10% nitric acid (v/v, H₂O) at 80° C. for 20 min, followed by rinsing with distilled/deionized water. The slides were then modified with (3-aminopropyl)trimethoxysilane, APTMS, by soaking in a solution of 10% APTMS (v/v, H₂O, pH 7) at 80° C. for 90 min, and then rinsed with distilled/deionized water. Finally, the modified slides were soaked in 10% glutaraldehyde (v/v, H₂O) at room temp for 60 min, rinsed with distilled/deionized water, and dried with a stream of nitrogen. Slides were used within 24 hr of preparation.

### Example 3

### Titanium Pre-Treatment

10 mm×10 mm×1 mm titanium coupons are out from a sample of titanium sheet metal via shearing. The titanium coupons are sonicated at 120% power for 20 min in ethanol, followed by 20 min, in acetone, and then 20 mins in deionized water. The coupons are then etched in a 50% (v/v) concentrated sulfuric acid solution in water for 30 min at 60° C. Following thorough rinsing with deionized water, the etched titanium coupons are then sonicated in deionized water for 20 min. Then, they are placed in a “piranha” solution (7.5 mL of conc. sulfuric acid: 2.5 mL of 30% hydrogen peroxide) for 10 minutes (for surface hydroxylation). The coupons are then rinsed multiple times with deionized water and then sonicated (2×) in deionized water for 10 min. Coupons are stored in deionized water. Prior to use, they are dried under flow of nitrogen,

### Example 4

### 30% BAP-3/MTMOS Film Synthesis

Absolute ethanol (211 μL) and methyltrimethoxysilane, MTMOS (140 μL) were added to a 1.5 mL polypropylene centrifuge tube and vortexed for 10 s, BAP-3/NO solution (249 μL) and distilled/deionized water (60 μL) were added, vortexing 10 seconds after each addition. Sodium hydroxide (0.5 M, 10 μL) was added to the vial and the solution was vortexed 45 min. The sol solution (30 μL) was cast onto pre-treated 9×12.5 mm glass slides and spread carefully with a pipette tip to coat the slide evenly. The slides were dried for 80 min in a dessicator, and then stored in a sealed container inside a dessicator at −20° C. for 24 hr prior to use.

### Example 5

### Synthesis and Film Stability of NO-Releasing Coatings

Using the synthetic protocol described in Example 4, diazeniumdiolate-modified [3-(methylamino)propyl]trimethoxysilane (MAP-3/NO), butylamino-propyltrimethoxysilane (BAP-3/NO), N-(6-aminohexyl)aminopropyltrimethoxysilane (AHAP3/NO); and (3-trimethoxysilylpropyl)di-ethylenetriamine (DET3/NO) were each independently co-condensed with 10-30% (v/v) of butyltrimethoxysilane (BTMOS) or MTMOS. The sol was then coated onto pretreated glass slides and dried, and the stability of the resulting film was assessed. The results are shown in Table 1.

TABLE 1

| | | Volume % | |
|---|---|---|---|
| Aminosilane | Backbone | (Aminosilane/Total | |
| NONOate | Silane | Silane) | Film Stability |
| MAP-3/NO | BTMOS | 10 | Poor - never hardened |
| | BTMOS | 20 | Poor - never hardened |
| | BTMOS | 30 | Poor - never hardened |
| | MTMOS | 10 | Good |
| | MTMOS | 20 | Good |
| | MTMOS | 30 | Good |
| BAP-3/NO | BTMOS | 10 | Poor |
| | BTMOS | 20 | Good |
| | BTMOS | 30 | Good |
| | MTMOS | 10 | Poor |
| | MTMOS | 20 | Poor |
| | MTMOS | 30 | Good |
| AHAP-3/NO | BTMOS | 10 | Poor |
| | BTMOS | 20 | Good |
| | BTMOS | 30 | Good |
| | MTMOS | 10 | Poor |
| | MTMOS | 20 | Poor |
| | MTMOS | 30 | Poor |
| DET-3/NO | BTMOS | 10 | Poor - film dissolved |
| | BTMOS | 20 | Poor - film dissolved |
| | BTMOS | 30 | Poor - film dissolved |
| | MTMOS | 10 | Poor - film dissolved |
| | MTMOS | 20 | Poor - film dissolved |
| | MTMOS | 30 | Poor - film dissolved |

### Example 6

### NO Storage and Release

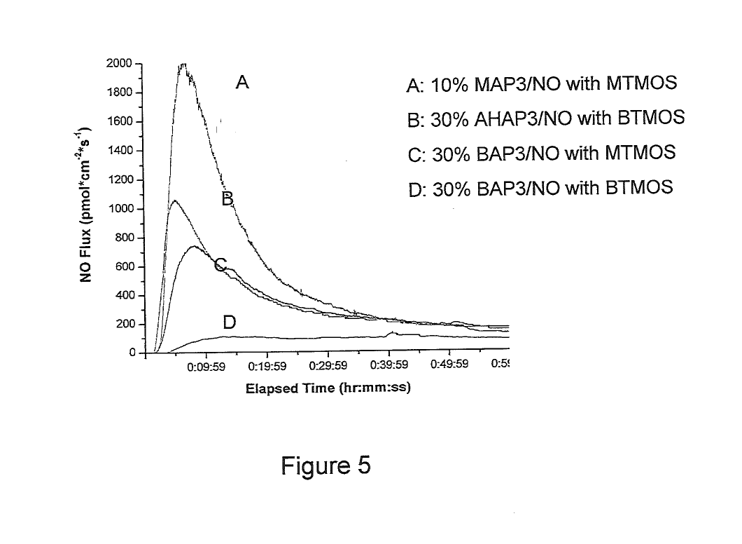

Select compositions of Example 4 were tested for NO storage and release characteristics including Total NO storage, half-life (t^{1/2}) and NO flux. The NO-release data was obtained using a NO chemiluminescence analyzer in pH 7.4 phosphate buffered saline at 37° C. The data obtained is shown in Table 2. The NO flux over time is shown in FIG. 5.

TABLE 2

| | | | Total NO | | |
|---|---|---|---|---|---|
| | Type of | Vol % of | Stored | | [NO]ₘ |
| Backbone | diazeniumdiolated | diazeniumdiolated | (μmol | t^{1/2} | (pmol · sec−1 · |
| alkylalkoxysilane | alkoxysilane | alkoxysilane | NO · cm⁻²) | (min) | cm⁻²) |
| MTMOS | MAP3/NO | 10 | 3.69 | 22 | 2050 |
| | AHAP3/NO | 30 | 4.07 | 202 | 1060 |
| | BAP3/NO | 30 | 4.06 | 170 | 690 |
| BTMOS | BAP3/NO | 30 | 2.91 | 395 | 130 |

### Example 7

### Titanium-Coated Films

Using the synthetic protocol described in Example 4, diazeniumdiolate-modified [3-(methylamino)propyl]trimethoxysilane (MAP-3/NO), butylamino-propyltrimethoxysilane (BAP-3/NO), N-(6-aminohexyl)aminopropyltrimethoxysilane (AHAP3/NO); N-(2-aminoethyl)-3-aminopropyltrimethoxysilane (AEAP3/NO) and (3-trimethoxysilylpropyl)di-ethylenetriamine (DET3/NO) were each independently co-condensed with 10-30% (v/v) of butyltrimethoxysilane (BTMOS), propyltrimethoxysilane (PTMOS) or MTMOS. The sol was then coated onto pretreated titanium coupons and dried, and the stability of the resulting film was assessed. The NO storage and release kinetics were also assessed. The results are shown in Table 3.

TABLE 3

| | | Mol % | | | | | |
|---|---|---|---|---|---|---|---|
| | | Aminosilane | Solvent | Spin | | | |
| Aminosilane | Backbone | of Total | Spread | Coated | Drying | NO-release | Film |
| NONOate | Silane | Silane | Cast | Films | Conditions | (μmoles NO/cm²) | Stability |
| MAP-3/NO | BTMOS | 10 | X | | 25° C./D/2 d | ND | Poor |
| | BTMOS | 20 | X | | 25° C./D/2 d | ND | Poor |
| | BTMOS | 30 | X | | 25° C./D/2 d | ND | Poor |
| | PTMOS | 10 | X | | 60° C./C/2 d | 0.6 | Good |
| | PTMOS | 20 | X | | 60° C./C/2 d | 2.9 | Good |
| | PTMOS | 30 | X | | 60° C./C/2 d | 7.3 | Good |
| | MTMOS | 10 | X | | 25° C./D/2 d | 4.0 | Good |
| | MTMOS | 20 | X | | 25° C./D/2 d | ND | Poor |
| | MTMOS | 30 | X | | 25° C./D/2 d | ND | Poor |
| BAP-3/NO | BTMOS | 10 | X | | 25° C./D/2 d | ND | Poor |
| | BTMOS | 20 | X | | 25° C./D/2 d | — | Good |
| | BTMOS | 30 | X | | 25° C./D/2 d | 2.9 | Good |
| | PTMOS | 10 | X | | 60° C./C/2 d | 0.2 | Good |
| | PTMOS | 20 | X | | 60° C./C/2 d | 3.5 | Good |
| | PTMOS | 30 | X | | 60° C./C/2 d | 5.2 | Good |
| | MTMOS | 10 | X | | 25° C./D/2 d | 3.7 | Good |
| | MTMOS | 20 | X | | 25° C./D/2 d | ND | Poor |
| | MTMOS | 30 | X | | 25° C./D/2 d | ND | Poor |
| AHAP-3/NO | BTMOS | 10 | X | | 60° C./C/2 d | ND | Poor |
| | BTMOS | 20 | X | | 60° C./C/2 d | — | Good |
| | BTMOS | 30 | X | | 60° C./V/2 d | 7.0 | Good |
| | PTMOS | 30 | X | | 60° C./V/2 d | 8.2 | Good |
| | MTMOS | 10 | X | | 60° C./V/2 d | ND | Poor |
| | MTMOS | 20 | X | | 60° C./V/2 d | ND | Poor |
| | MTMOS | 30 | X | | 60° C./V/2 d | ND | Poor |
| | BTMOS | 30 | | X | 60° C./V/2 d | 0.6 | Good |
| | BTMOS | 40 | | X | 60° C./V/2 d | 2.8 | Good |
| | BTMOS | 50 | | X | 60° C./V/2 d | ND | Poor |
| | PTMOS | 30 | | X | 60° C./V/2 d | 0.7 | Good |
| | MTMOS | 30 | | X | 60° C./V/2 d | ND | Poor |
| AEAP-3/NO | BTMOS | 30 | X | | 60° C./V/2 d | 11.2 | Good |
| | PTMOS | 30 | X | | 60° C./V/2 d | 10.9 | Good |
| | MTMOS | 30 | X | | 60° C./V/2 d | ND | Poor |
| | BTMOS | 30 | | X | 60° C./V/2 d | 0.9 | Good |
| | PTMOS | 30 | | X | 60° C./V/2 d | 0.9 | Good |
| | MTMOS | 30 | | X | 60° C./V/2 d | ND | Poor |
| DET-3/NO | BTMOS | 10 | X | | 25° C./B/3 h | ND | Poor |
| | BTMOS | 20 | X | | 25° C./B/3 h | ND | Poor |
| | BTMOS | 30 | X | | 25° C./B/3 h | ND | Poor |
| | MTMOS | 10 | X | | 25° C./B/3 h | ND | Poor |
| | MTMOS | 20 | X | | 25° C./B/3 h | ND | Poor |
| | MTMOS | 30 | X | | 25° C./B/3 h | ND | Poor |

Drying Key: Temperature/Drying Method/Drying Duration:
C = Conventional Oven,
V = Vacuum Oven,
B = Bench,
D = Desiccator

### Example 8

### Fluorosilane Films

37.9 μL (0.20 mmol) isobutyltrimethoxy silane, 50 μL methanol were mixed, 5 μL of 0.1M HCl was added, and the resulting sol was allowed to react for 30 min. Then, 18 μl of 0.5M KOH is added, the sol is again briefly mixed and then 106 μL of a 25% v/v AEAP-3 charging solution in methanol is added [essentially: 79.5 μL methanol, and 26.5 μL AEAP-3/NO (0.11 mmol AEAP-3/NO)]. Afterwards, 29.2 μL of heptadecafluoro-1,1,2,2-tetrahydrodecyl)trimethoxysilane (0.07 mmol) was added. This is allowed to react for 1 hour while vortexing. Following centrifuging at 11,000 rpm for 10 sec, 64 μL was dispensed on 1 cm×1 cm titanium substrates that were spincoated at 2000 rpm for 10 sec,

### Example 9

### Prehydrolysis of Backbone Alkoxysilane

In this example, AEAP-3 (30 mol %) is used as the aminosilane and PTMOS (70 mol %) is used as the backbone silane. In a microcentrifuge tube, 45 μL of propyltrimethoxysilane (0.26 mmol), 50 μL of methanol are combined and mixed briefly. Following, 5 μL of 0.1M HCl is added, and allowed to react while agitating via a vortex for 30 min. Then, 18 μL of 0.5M KOH is added, the sol is again briefly mixed and then 106 μL of a 25% v/v AEAP-3 charging solution in methanol is added [essentially: 79.5 μL methanol, and 26.5 μL AEAP-3/NO (0.11 mmol AEAP-3/NO)]. This is allowed to react for 1 hour while vortexing. Prior to casting, it is centrifuged for 10 sec at 11,000 rpm,

### Example 10

### Post-Charged 30% AEAP-3/70% BTMOS (Comparative Example)

10 mm×10 mm×1 mm titanium coupons are cut from a sample of titanium sheet metal via shearing. The titanium coupons are sonicated at 120% power for 20 min in ethanol, followed by 20 mins in acetone, and then 20 mins in deionized water. The coupons are then etched in a 50% (v/v) cone sulfuric acid solution in water for 30 min at 60° C.

Following copious rinsing with deionized water, the etched titanium coupons are then sonicated in deionized water for 20 min. Then, they are placed in a “piranha” solution (7.5 mL of cone sulfuric acid: 2.5 mL of 30% hydrogen peroxide) for 10 minutes (for surface hydroxylation). Following, coupons are rinsed multiple times with deionized water and then sonicated (2×) in deionized water for 10 min. Coupons are stored in deionized water. Prior to use, they are dried under flow of nitrogen.

In a microcentrifuge tube, 94.5 μL isobutyltrimethoxysilane (BTMOS), 170 μL methanol, 30 μL H₂O, and 5 μL 0.5M hydrochloric acid were combined. Following 1 hr of vortexing, 56.5 AEAP-3 was added and the sol was vortexed for an additional 1 hr.

64 μL of the resulting sol was dispensed onto the titanium coupons. The sols were immediately spin-coated at 1000 rpm for 10 s. Films were allowed to dry on the benchtop for 30 min and then placed in a 60° C. oven for 48 hr.

The films were placed in a Parr hydrogenation bomb and purged with 10 atm argon (3×), and then held under 10 atm argon for 10 min (3×). After the three 10 min purge cycles, the bomb was filled with NO to 10 atm and held for 48 hr. Following the 48 hour NO exposure, films were again purged with 10 atm argon (3×) and then held under 10 atm argon for 10 min (3×). Films stored under nitrogen at −20° C. until analysis,

### Example 11

### Pre-Charged 30% AEAP-3/70% BTMOS

10 mm×10 mm×1 mm titanium coupons are cut from a sample of titanium sheet metal via shearing. The titanium coupons are sonicated at 120% power for 20 min in ethanol, followed by 20 mins in acetone, and then 20 mins in deionized water. The coupons are then etched in a 50% (v/v) cone sulfuric acid solution in water for 30 min at 60° C.

Following copious rinsing with deionized water, the etched titanium coupons are then sonicated in deionized water for 20 min. Then, they are placed in a “piranha” solution (7.5 mL of cone sulfuric acid: 2.5 mL of 30% hydrogen peroxide) for 10 minutes (for surface hydroxylation). Following, coupons are rinsed multiple times with deionized water and then sonicated (2×) in deionized water for 10 min. Coupons are stored in deionized water. Prior to use, they are dried under flow of nitrogen.

In a microcentrifuge tube, 106 μL isobutyltrimethoxysilane (BTMOS), 226 μL of a 25% v/v AEAP/NO charged solution in methanol, and 36 μL 0.5M KOH were combined. The mixture was allowed to react while vortexing for 30 min, 64 μL of the resulting sol was dispensed onto the titanium coupons. The sols were immediately spin-coated at 1000 rpm for 10 s. Following spincoating, the films were held under N₂ for 15 min at room temperature. The temperature was then ramped to 60° C. and held for an additional 30 minutes, while still under N₂ flow. While maintaining a 60° C. temperature, vacuum was applied for 48 hours. Following drying, films were stored under N₂ at −20° C.

### Comparative Example

Six 1 cm×1 cm×1 mm titanium coupons were placed in hexane, and ultrasonicated for 15 min. They were then transferred to 2-propanol and ultrasonicated for an additional 15 min. After thoroughly rinsing with deionized water, the coupons were ultrasonicated in 1M NaOH for 15 min. The coupons were again rinsed with deionized water, and dried under vacuum at >40° C.

In a small vial, 1.0 g of N-(2-aminoethyl)-3-aminopropyltrimethoxysilane (AEAP-3) was combined with 1.4 g acetonitrile and 0.6 g tetrahydrofuran (THF) and allowed to mix, to ensure that the aminosilane was fully dissolved. Each titanium coupon was submerged in the resulting solution for 10 s, and then withdrawn. Any excess solvent was allowed to drain, then the coated coupons were kept at room temperature in ambient conditions for 30 minutes. Afterwards, the coated coupons were placed under vacuum at >40° C. overnight.

After drying in the vacuum oven, each coated coupon was placed in a small glass vial along with 4 mL of THF. The vials were placed in a Parr Hydrogenation bomb, purged w 4 atm argon (10×), and then 4 atm NO (10×) before finally being held under NO at a pressure of 4 atm for 48 hr.

After 48 hr, the hydrogenation bomb was purged with 4 atm argon (10×). The THF was decanted, and the coated coupons were rinsed with 4 mL THF (1×) and then 4 mL diethyl ether (3×). They were then dried under a stream of nitrogen, and analyzed using a Sievers 280 Nitric Oxide Analyzer.

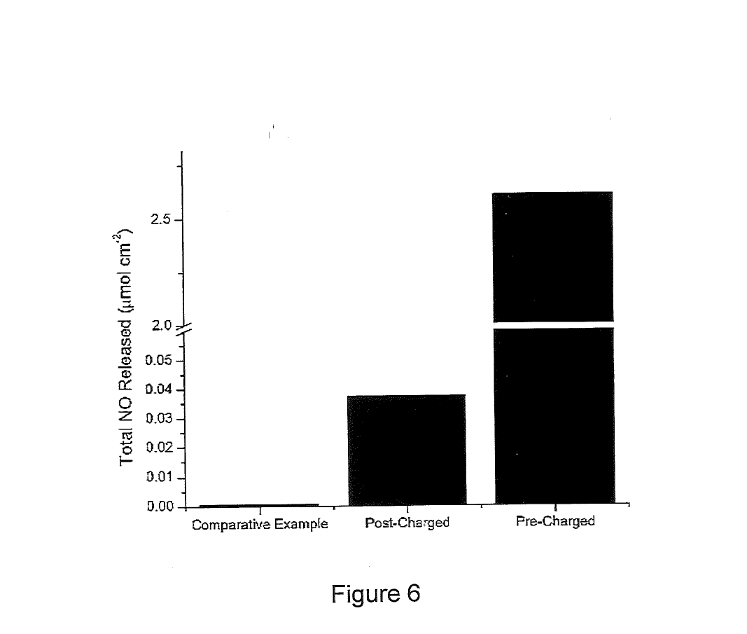

FIG. 6 compares the NO loading of the pre-charged coatings of Example 11, the post-charged coatings of Example 10 and the Comparative Example. As can be seen in FIG. 6, coatings according to some embodiments of the invention may have significantly higher NO loadings compared to other diazeniumdiolate-modified films.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1.-25. (canceled)

26. A method of producing a NO-releasing sol-gel coating comprising:
(a) co-condensing a sol precursor solution comprising a backbone alkoxysilane and a diazeniumdiolate-modified alkoxysilane in a solvent to form a sol;
(b) coating a substrate with the sol; and
(c) drying the sol to form the NO-releasing sol-gel coating.

27. The method of claim 25, wherein the sol precursor solution further comprises a base catalyst.

28. The method of claim 27, wherein the base catalyst comprises at least one compound selected from the group consisting of ammmonia, alkali metal hydroxides, fluorides (NaF) and an organic base.

29. The method of claim 26, wherein the sol precursor solution further comprises a multifunctional alkoxysilane that comprises at least one functionality that provides to the sol-gel coating at least one additional property selected from the group consisting of anti-corrosive activity, anti-inflammatory activity, anti-microbial activity, anti-oxidative activity, additional cross-linking functionality, surface charge, hydrophilicity and hydrophobicity.

30. The method of claim 29, wherein the multifunctional alkoxysilane has the formula R′R″R′″SiOR, wherein R is H, alkyl or substituted alkyl, R′, R″ and R′″ are each independently a substituted or unsubstituted alkyl, a substituted or unsubstituted aryl, a substituted or unsubstituted alkylaryl, a substituted or unsubstituted arylalkyl or an organic moiety that provides at least one additional property to the sol-gel coating, wherein at least one or R′, R″ and R′″ is an organic moiety that provides at least one additional property to the sol-gel coating.

31. The method of claim 26, wherein the backbone alkoxysilane comprises at least one alkoxysilane selected from the group consisting of methyltrimethoxysilane (MTMOS), butyltrimethoxysilane (BTMOS), butyltriethoxysilane (BTEOS), propyltrimethoxysilane (PTMOS) and octadecyltrimethoxysilane (ODTMOS).

32. The method of claim 26, wherein the diazeniumdiolate-modified alkoxysilane comprises at least one diazeniumdiolate-modified alkoxysilane selected from the group consisting of N-(6-aminohexyl)aminopropyltrimethoxysilane (AHAP3); N-(2-aminoethyl)-3-aminopropyltrimethoxysilane (AEAP3); (3-trimethoxysilylpropyl)diethylenetriamine (DET3); (aminoethylaminomethyl)phenethyltrimethoxysilane (AEMP3); [3-(methylamino)propyl]trimethoxysilane (MAP3); N-butylamino-propyltrimethoxysilane(n-BAP3); t-butylamino-propyltrimethoxysilane(t-BAP3); N-ethylaminoisobutyltrimethoxysilane(EAiB3); N-phenylamino-propyltrimethoxysilane (PAP3); and N-cyclohexylaminopropyltrimethoxysilane (cHAP3).

33. The method of claim 26, wherein the substrate is coated by dip-coating, spread-coating, spray coating or combinations thereof.

34. The method of claim 26, wherein the substrate is coated with two or more layers of the sol.

35. The method of claim 26, wherein the substrate is further coated with an additional coating material that is not the sol.

36. The method of claim 26, wherein co-condensing the sol precursor solution comprises co-condensing backbone alkoxysilane in the absence of the diazeniumdiolate-modified alkoxysilane for a specified time, and then adding the diazeniumdiolate-modified alkoxysilane to form the sol.

37. The method of claim 29, wherein the at least one additional property is hydrophobicity and the multifunctional alkoxysilane comprises at least one of a linear alkyl alkoxysilane, a branched alkylalkoxysilane, a cyclic alkylalkoxysilanes, a substituted and unsubstituted phenyl alkoxysilane, and a fluorinated alkoxysilane.

38. The method of claim 37, wherein the multifunctional alkoxysilane comprises at least one of heptadecafluoro-1,1,2,2-tetrahydrodecyl)triethoxysilane, (3,3,3-trifluoropropyl)trimethoxysilane, (perfluoroalkyl)ethyltriethoxysilane, nonafluorohexyltrimethoxysilane, nonafluorohexyltriethoxysilane, (tridecafluoro-1,1,2,2-tetrahydrooctyl)triethoxysilane and (tridecafluoro-1,1,2,2-tetrahydrooctyl)trimethoxysilane.

39. The method of claim 29, wherein the multifunctional alkoxysilane has the formula R′R″R′″SiOR, wherein R is H, alkyl or substituted alkyl, R′, R″ and R′″ are each independently a substituted or unsubstituted alkyl, a substituted or unsubstituted aryl, a substituted or unsubstituted alkylaryl, a substituted or unsubstituted arylalkyl or an organic moiety that provides at least one additional property to the sol-gel coating, wherein at least one or R′, R″ and R′″ is an organic moiety that provides at least one additional property to the sol-gel coating.

40. The method of claim 39, wherein R₃ comprises at least one alkoxysilane selected from the group consisting of a fluorinated alkoxysilane, a cationic or anionic alkoxysilane and a pegylated alkoxysilane.

41. The method of claim 26, wherein the coating has an NO storage greater than 0.01 μmol NO·cm⁻².

42. The method of claim 26, wherein the volume of diazeniumdiolate-modified alkoxysilane is in a range of about 10 to about 40% of the total alkoxysilane volume.

43. The method of claim 26, wherein the volume of backbone alkoxysilane is in a range of about 10 to about 30 percent of the total alkoxysilane volume.

44. The method of claim 26, wherein the coating has an NO storage greater than 7.0 μmol NO·cm⁻².
